(19) United States
(12) Patent Application Publication
(10) Pub. No.: US 2005/0143441 A1
(43) Pub. Date: Jun. 30, 2005

(54) Novel medical combination treatment of obesity involving 4,5-dihydro-1H-pyrazole derivatives having CB1-antagonistic activity

(76) Inventors: Jochen Antel, Bad Munder (DE); Gunter Krause, Burgdorf (DE); Peter-Colin Gregory, Hannover (DE); Michael Wurl, Garbsen (DE); Harald Waldeck, Isernhagen (DE); Josephus Hubertus Lange, Almere (NL); Cornelis Gerrit Kruse, Amersfoort (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/005,486

(22) Filed: Dec. 7, 2004

Related U.S. Application Data
(63) Continuation-in-part of application No. 10/972,006, filed on Oct. 25, 2004.
(60) Provisional application No. 60/513,994, filed on Oct. 27, 2003.

(51) Int. Cl. A61K 31/4152; A61K 31/365

(52) U.S. Cl. 514/406; 514/449

(57) ABSTRACT

The present invention relates to a novel medical use of e.g. 4,5-dihydro-1H-pyrazole compounds which are potent antagonists of the cannabis CB₁-receptor in combination with lipase inhibitors. Said compounds are particularly suitable in combination with lipase inhibitors in the manufacture of medicaments for the treatment and/or prophylaxis of obesity in adolescent or in juvenile patients and/or for the treatment and/or prophylaxis of drug induced obesity in juvenile as well as in adolescent patients. The compounds have the general formula (I) 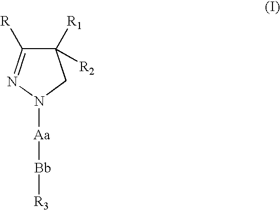
wherein the group Bb represents sulfonyl or carbonyl, and the substituents R, R₁, R₂ and R₃, and the group Aa are defined as shown in the description. Preferred lipase inhibitors are orlistat, panclicins, ATL-962 and/or lipstatin.

### CROSS REFERENCE TO RELATED APPLICATIONS

This application is a continuation-in-part of U.S. application Ser. No. 10/972,006, filed on Oct. 25, 2004, which claims the benefit of U.S. Provisional Application No. 60/513,994, filed on Oct. 27, 2003; the contents of the foregoing applications are incorporated herein by reference.

The present invention relates to a novel therapeutic and/or prophylactic treatment of obesity by administering a combination of a 4,5-dihydro-1H-pyrazole derivative having CB₁-antagonistic activity together with a further active principle, and to pharmaceutical compositions containing at least one of these CB₁-antagonistic compounds in combination with said further active principle for the treatment and/or prophylaxis of obesity. The combination provided by the present invention of a 4,5-dihydro-1H-pyrazole showing potent Cannabis-1 (CB₁) receptor antagonistic activity with said further active principle are of particular utility for treating of obesity.

Cannabinoids are present in the Indian hemp Cannabis Sativa L. and have been used as medicinal agents for centuries (Mechoulam, R.; Feigenbaum, J. J. Prog. Med. Chem. 1987, 24, 159). However, only within the past ten years the research in the cannabinoid area has revealed pivotal information on cannabinoid receptors and their (endogenous) agonists and antagonists. The discovery and the subsequent cloning of two different subtypes of Cannabinoid receptors (CB₁ and CB₂) stimulated the search for novel cannabinoid receptor antagonists (Munro, S.; Thomas, K. L.; Abu-Shaar, M. Nature 1993, 365, 61. Matsuda, L. A.; Bonner, T. I. Cannabinoid Receptors, Pertwee, R. G. Ed. 1995, 117, Academic Press, London). In addition, pharmaceutical companies became interested in the development of cannabinoid drugs for the treatment of diseases connected with disorders of the cannabinoid system. The wide distribution of CB₁ receptors in the brain, in combination with the strictly peripheral localization of the CB₂ receptor, makes the CB₁ receptor a very interesting molecular target for CNS-directed drug discovery in several areas of medical indications, e.g. psychiatric and neurological disorders are described in the state of the art as being of interest (Consroe, P. Neurobiology of Disease 1998, 5, 534. Pop, E. Curr. Opin. In CPNS Investigational Drugs 1999, 1, 587. Greenberg, D. A. Drug News Perspect. 1999, 12, 458).

From the international patent application WO 01/70700 4,5-dihydro-1H-pyrazole compounds are known which exhibit potent and selective cannabis CB₁-receptor antagonistic activity. These compounds have the formula (I) defined below, and have been suggested for use in the treatment of psychiatric disorders such as psychosis, anxiety, depression, attention deficits, memory disorders and appetite disorders, obesity, neurological disorders such as dementia, distonia, Parkinson's disease, Alzheimer's disease, epilepsy, Huntington's disease, Tourette's syndrome, cerebral ischemia, as well as for the treatment of pain disorders and other CNS-diseases involving cannabinoid neurotransmission, and in the treatment of gastrointestinal disorders and cardiovascular disorders.

It has now surprisingly been found that CB₁ antagonistic compounds (CB₁ antagonists), in particular 4,5-dihydro-1H-pyrazole derivatives which are potent and selective antagonists of the cannabis CB₁-receptor, as well as prodrugs, tautomers and salts thereof, due to their unique pharmacological profile are particularly suited in combination with at least one lipase inhibiting compound (lipase inhibitor) for the use in the manufacture of a medicaments for the treatment and/or prophylaxis of obesity, including in particular the treatment and/or prophylaxis of obesity in juvenile patients and/or drug induced obesity in juvenile as well as adolescent patients. In this regard combinations of at least one CB₁ antagonistic compound with at least one lipase inhibiting compound are highly valuable in providing medicaments for the treatment and/or prophylaxis of obesity in general, e.g. in adolescent patients of any age, and particularly also in paediatric or juvenile obesity, and also in drug induced obesity in adolescent and juvenile patients.

Therefore, the invention pertains to the combination of a CB₁ antagonistic compound, in particular a 4,5-dihydro-1H-pyrazole derivative which is a potent and selective antagonist of the cannabis CB₁-receptor, or a prodrug, tautomer or salt thereof, with at least one lipase inhibiting compound.

Prodrugs are bioreversible derivatives of drug molecules used to overcome some barriers to the utility of the parent drug molecule. These barriers include, but are not limited to, solubility, permeability, stability, presystemic metabolism And targeting limitations (J. Stella, “Prodrugs as therapeutics”, Expert Opin. Ther. Patents, 14(3), 277-280, 2004). Pro-drugs, i.e., compounds which when administered to humans by any known route, are metabolized to compounds having a formula set forth herein, belong to the invention. For example, this includes compounds with primary or secondary amino or hydroxy groups. Such compounds can be reacted with organic acids to yield compounds having a formula set forth herein wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone. A pro-drug is an inactive compound, which when administered is converted into an active form. See Medicinal Chemistry: Principles and Practice, 1994, ISBN 0-85186-494-5, Ed.: F. D. King, p. 215.

As stated above, the compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

In particular the present invention is based on the surprising finding that the 4,5-dihydro-1H-pyrazole derivatives of the formula (I) which are potent and selective antagonists of the cannabis CB₁-receptor, prodrugs thereof, tautomers thereof and salts thereof. 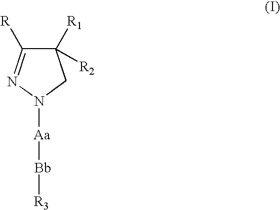
wherein
R and R₁ are the same or different and represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
Aa represents one of the groups (i), (ii), (iii), (iv) or (v) 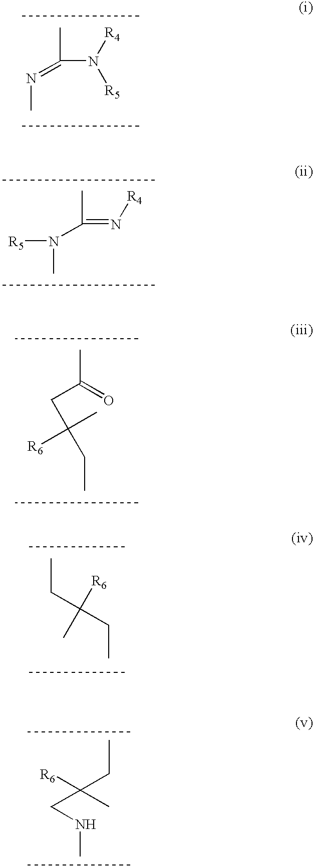
wherein
   R₄ and R₅ independently of each other represent hydrogen or C₁₋₈ branched or unbranched alkyl or C₃₋₈ cycloalkyl or R₄ represents acetamido or dimethylamino or 2,2,2-trifluoroethyl or phenyl or pyridyl with the proviso that R₅ represents hydrogen
   R₆ represents hydrogen or C₁₋₃ unbranched alkyl
Bb represents sulfonyl or carbonyl,
R₃ represents benzyl, phenyl, thienyl or pyridyl which may be substituted with 1, 2 or 3 substituents Y, which can be the same or different, or R₃ represents C₁₋₈ branched or unbranched alkyl or C₃₋₈ cycloalkyl, or R₃ represents naphtyl due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (I) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.

The outstanding unique pharmacological profile of CB₁ antagonistic compounds (CB₁ antagonists), and in particular of the 4,5-dihydro-1H-pyrazole derivatives as defined in formula (I) which are potent and selective antagonists of the cannabis CB₁-receptor, as well as prodrugs, tautomers and salts thereof, includes particularly high safety and tolerability also in combination with other drugs, in particular in combination with lipase inhibiting compounds according to the present invention. Thus the CB₁ antagonistic compounds in combination with lipase inhibiting compounds are particularly suitable also in patient groups with enhanced need of safety and tolerability, in particular such as juvenile patients and/or patients subject to long term treatment, e.g. in drug induced obesity.

This safety and tolerability of CB₁ antagonistic compounds in combination with lipase inhibiting compounds is advantageous in the treatment and/or prophylaxis of obesity in those patient populations where a single treatment is not sufficiently effective and a combination treatment and/or prophylaxis involving different medical or metabolic mechanisms is desired or required for achieving and stabilizing a defined degree of weight loss.

Hence, combination of CB₁ antagonistic compounds in combination with lipase inhibiting compounds according to the present invention is expected to be very is advantageous in the treatment and/or prophylaxis of obesity in general, e.g. of obesity in adolescent patients of any age, and particularly also in pediatric or juvenile obesity, and in drug induced obesity.

The CB₁ receptor modulating activity of the compounds of the invention makes them particularly useful in the treatment of obesity, juvenile obesity and drug induced obesity, when used in combination with lipase inhibitors. Specific examples of lipase inhibiting compounds which can be used in such combination preparations are (but not restricted to) the synthetic lipase inhibitor orlistat, panclicins, lipase inhibitors isolated from micro organisms such as lipstatin (from Streptomyces toxytricini), ebelactone B (from Streptomyces aburaviensis), synthetic derivatives of these compounds, 2-oxy-4H-3,1-benzoxazin-4-one derivatives like ATL-962 and structurally related compounds, 2-amino-4H-3,1-benzoxazin-4-one derivatives, as well as extracts of plants known to possess lipase inhibitory activity, for instance extracts of Alpinia officinarum Hance or compounds isolated from such extracts like 3-methylethergalangin (from A. officinarum). The lipase inhibiting compound may also be a lipase inhibiting polymer. These lipase inhibiting compounds and their manufacture are well known in the state of the art.

The affinity of the compounds of formula (I) for cannabinoid CB₁ receptors is described in the WO 01/70700, e.g. it was determined using membrane preparations of Chinese hamster ovary (CHO) cells in which the human cannabis CB₁ receptor is stably transfected in conjunction with [3H]CP-55,940 as radioligand. After incubation of a freshly prepared cell membrane preparation with the [3H]-ligand, with or without addition of compounds of the invention, separation of bound and free ligand was performed by filtration over glass fibre filters. Radioactivity on the filter was measured by liquid scintillation counting.

The cannabinoid CB₁ antagonistic activity of compounds of formula (I) is also described in the WO 01/70700, and was determined by functional studies using CHO cells in which human cannabinoid CB₁ receptors are stably expressed. Adenylyl cyclase was stimulated using forskolin and measured by quantifying the amount of accumulated cyclic AMP. Concomitant activation of CB₁ receptors by CB₁ receptor agonists (e.g. CP-55,940 or (R)-WIN-55,212-2) can attenuate the forskolin-induced accumulation of cAMP in a concentration-dependent manner. This CB₁ receptor-mediated response can be antagonized by CB₁ receptor antagonists such as the compounds used in the present invention.

The whole content of the international patent application WO 01/70700 is incorporated by reference into the present application regarding the disclosure of the CB₁ antagonistic compounds used according to the present invention in combination with lipase inhibitors.

At least one centre of chirality is present (at the C₄ position of the 4,5-dihydro-1H-pyrazole moiety) in the compounds of the formula (I). The invention relates both to the novel use of racemates, mixtures of diastereomers and the individual stereoisomers of the compounds having formula (I). The invention also relates both to the novel use of the E isomer, Z isomer and E/Z mixtures of compounds having formula (I) wherein Aa has the meaning (i) or (ii) as described herein above.

According to one embodiment of the invention compound having formula (I) are used, wherein R is the group 4-chlorophenyl, R₁ is phenyl, R₂ is hydrogen, Aa is the group (i) wherein R₄ is hydrogen and R₅ is methyl, Bb is sulfonyl, and R₃ represents 4-chlorophenyl, and salts thereof. The compound having formula (I) used according to the invention may be a levorotatory enantiomer.

The compounds of formula (I) used in the present the invention can be obtained according to known methods. A suitable synthesis for the compounds used according to the present invention is described for compounds of formula (I) in the international patent application WO 01/70700. For example compounds having formula (III) (vide infra), wherein R₂ represents hydrogen can be also obtained according to methods known, for example: a) EP 0021506; b) DE 2529689.

Example compounds of formula (I) having been prepared according to WO 01/70700 and being investigated include the e.g. the following compounds:
1) 3-(4-Chlorophenyl)-4,5-dihydro-4-hydroxy-4-phenyl-1H-pyrazole
2) 3-(4-Chlorophenyl)-4,5-dihydro-N-((4-fluorophenyl)sulfonyl)-4-phenyl-1H-pyrazole-1-carboxamidine
3) 4,5-Dihydro-N-((4-fluorophenyl)sulfonyl)-3-(4-methoxyphenyl)-4-(4-methoxy-phenyl)-1H-pyrazole-1-carboxamidine
4) 4,5-Dihydro-3-(4-methoxyphenyl)-4-(4-methoxyphenyl)-N-((4-methoxy-phenyl)sulfonyl)-1H-pyrazole-1-carboxamidine
5) 3-(4-Chlorophenyl)-4,5-dihydro-4-phenyl-N-((2,4,6-trimethylphenyl)sulfonyl)-1H-pyrazole-1-carboxamidine
6) 3-(4-Chlorophenyl)-4,5-dihydro-N-((4-fluorophenyl)sulfonyl)-4-hydroxy-4-phenyl-1H-pyrazole-1-carboxamidine
7) 3-(4-Chlorophenyl)-4,5-dihydro-N-(1-naphtoyl)-4-phenyl-1H-pyrazole-1-carboxamidine
8) 3-(4-Chlorophenyl)-4,5-dihydro-4-phenyl-N-(2-pyridoyl)-1H-pyrazole-1-carboxamidine
9) N¹,N¹-Dimethyl-N²-((4-chlorophenyl)sulfonyl)-3-(4-chlorophenyl)-4,5-dihydro-4-phenyl-1H-pyrazole-1-carboxamidine
10) N-Methyl-N′-((4-chlorophenyl)sulfonyl)-3-(4-chlorophenyl)-4,5-dihydro-4-(3-pyridyl)-1H-pyrazole-1-carboxamidine
11) N-Methyl-N′-((4-chlorophenyl)sulfonyl)-3-(4-chlorophenyl)-4,5-dihydro-4-(4-pyridyl)-1H-pyrazole-1-carboxamidine
12) N¹,N¹-Dimethyl-N²-((4-chlorophenyl)sulfonyl)-3-(4-chlorophenyl)-4,5-dihydro-4-hydroxy-4-phenyl-1H-pyrazole-1-carboxamidine
13) N-Ethyl-N-((4-chlorophenyl)sulfonyl)-3-(4-chlorophenyl)-4,5-dihydro-4-hydroxy-4-phenyl-1H-pyrazole-1-carboxamidine
14) N-Methyl-N′-(3-(trifluoromethyl)benzoyl)-3-(4-chlorophenyl)-4,5-dihydro-4-phenyl-1H-pyrazole-1-carboxamidine
15) N-Methyl-N′-((4-chlorophenyl)sulfonyl)-3-(4-chlorophenyl)-4,5-dihydro-4-phenyl-1H-pyrazole-1-carboxamidine
16) N-Methyl-N′-((3-chlorophenyl)sulfonyl)-3-(4-chlorophenyl)-4,5-dihydro-4-phenyl-1H-pyrazole-1-carboxamidine
17) N-Methyl-N′-((4-chlorophenyl)sulfonyl)-3-(5-chloro-2-thienyl)-4,5-dihydro-4-phenyl-1H-pyrazole-1-carboxamidine
18) N-Propyl-N′-((4-fluorophenyl)sulfonyl)-3-(4-chlorophenyl)-4,5-dihydro-4-phenyl-1H-pyrazole-1-carboxamidine
19) N-(2-Propyl)-N′-((4-fluorophenyl)sulfonyl)-3-(4-chlorophenyl)-4,5-dihydro-4-phenyl-1H-pyrazole-1-carboxamidine
20) N-Methyl-N′-((2-propyl)sulfonyl)-3-(4-chlorophenyl)-4,5-dihydro-4-phenyl-1H-pyrazole-1-carboxamidine
21) N-(2-Propyl)-N′-((4-chlorophenyl)sulfonyl)-3-(4-pyridyl)-4,5-dihydro-4-phenyl-1H-pyrazole-1-carboxamidine
22) N¹-Ethyl-N¹-methyl-N²-((4-chlorophenyl)sulfonyl)-3-(4-chlorophenyl)-4,5-dihydro-4-phenyl-1H-pyrazole-1-carboxamidine
23) N¹-Ethyl-N¹-methyl-N²-((4-fluorophenyl)sulfonyl)-3-(4-chlorophenyl)-4,5-dihydro-4-phenyl-1H-pyrazole-1-carboxamidine
24) N¹,N¹-Dimethyl-N²-((4-(trifluoromethyl)phenyl)sulfonyl)-3-(4-chlorophenyl)-4,5-dihydro-4-phenyl-1H-pyrazole-1-carboxamidine
25) N¹,N¹-Dimethyl-N²-((3-methylphenyl)sulfonyl)-3-(4-chlorophenyl)-4,5-dihydro-4-phenyl-1H-pyrazole-1-carboxamidine
26) N¹, N¹-Dimethyl-N²-((3-methoxyphenyl)sulfonyl)-3-(4-chlorophenyl)-4,5-dihydro-4-phenyl-1H-pyrazole-1-carboxamidine
27) N-Ethyl-N′-((4-chlorophenyl)sulfonyl)-3-(4-chlorophenyl)-4,5-dihydro-4-phenyl-1H-pyrazole-1-carboxamidine
28) N-Dimethylamino-N′-((4-chlorophenyl)sulfonyl)-3-(4-chlorophenyl)-4,5-dihydro-4-phenyl-1H-pyrazole-1-carboxamidine
29) N-Methyl-N′-((4-(trifluoromethyl)phenyl)sulfonyl)-3-(4-chlorophenyl)-4,5-dihydro-4-phenyl-1H-pyrazole-1-carboxamidine
30) N¹,N¹-Dimethyl-N²-((2-methylphenyl)sulfonyl)-3-(4-chlorophenyl)-4,5-dihydro-4-phenyl-1H-pyrazole-1-carboxamidine
31) N-Methyl-N′-((2,4-difluorophenyl)sulfonyl)-3-(4-chlorophenyl)-4,5-dihydro-4-phenyl-1H-pyrazole-1-carboxamidine
32) N-Acetamido-N′-((4-chlorophenyl)sulfonyl)-3-(4-chlorophenyl)-4,5-dihydro-4-phenyl-1H-pyrazole-1-carboxamidine
33) N-(2,2,2-Trifluoroethyl)-N′-((4-chlorophenyl)sulfonyl)-3-(4-chlorophenyl)-4,5-dihydro-4-phenyl-1H-pyrazole-1-carboxamidine
34) N-(2-Pyridyl)-N′-((4-chlorophenyl)sulfonyl)-3-(4-chlorophenyl)-4,5-dihydro-4-phenyl-1H-pyrazole-1-carboxamidine
35) N-(4-Pyridyl)-N′-((4-chlorophenyl)sulfonyl)-3-(4-chlorophenyl)-4,5-dihydro-4-phenyl-1H-pyrazole-1-carboxamidine
36) N-Phenyl-N′-((4-chlorophenyl)sulfonyl)-3-(4-chlorophenyl)-4,5-dihydro-4-phenyl-1H-pyrazole-1-carboxamidine
37) 3-(4-Chlorophenyl)-1-[3-((4-chlorophenyl)sulfonyl)butanoyl]-4,5-dihydro-4-phenyl-1H-pyrazole
38) 3-(4-Chlorophenyl)-1-[3-(phenylsulfonyl)propanoyl]-4,5-dihydro-4-phenyl-1H-pyrazole
39) 3-(4-Chlorophenyl)-1-[3-((4-chlorophenyl)sulfonyl)propanoyl]-4,5-dihydro-4-phenyl-1H-pyrazole
40) 3-(4-Chlorophenyl)-4,5-dihydro-4-phenyl-1-[2-((3-(trifluoromethyl)phenyl)-sulfonyl)ethyl]-1H-pyrazole
41) 3-(4-Chlorophenyl)-1-[2-(benzylsulfonyl)ethyl]-4,5-dihydro-4-phenyl-1H-pyrazole
42) 3-(4-Chlorophenyl)-1-[2-((4-chlorophenyl)sulfonyl)ethyl]4,5-dihydro-4-phenyl-1H-pyrazole
43) 3-(4-Chlorophenyl)-1-[2-((4-chlorophenyl)sulfonyl)ethyl]4,5-dihydro-4-hydroxy-4-phenyl-1H-pyrazole
44) N-[2-(3-(4-Chlorophenyl)-4,5-dihydro-4-phenyl-1H-pyrazol-1-yl)ethyl]-3-(trifluoromethyl)benzenesulfonamide

Lipase inhibiting compounds used in the combinations according to the present invention may be any lipase inhibiting compound suitable for pharmaceutical use, e.g. in particular inhibitors of pancreatic lipases. Lipases are key enzymes in the digestive system which break down tri- and diglycerides, which are too large to be absorbed by the small intestine into fatty acids which can be absorbed. Since lipases are responsible for the hydrolysis of fat, a consequence of their inhibition is a reduction in fat hydrolysis and absorption. Therefore, inhibition of lipases results in a reduction in the absorption of fat. The lipase inhibiting compound is preferably the synthetic lipase inhibitor orlistat and structurally related compounds, 2-oxy-4H-3,1-benzoxazin-4-one derivatives like ATL-962 and structurally related compounds, 2-amino-4H-3,1-benzoxazin-4-one derivatives, lipase inhibitors isolated from micro organisms such as lipstatin, ebelactone B, or synthetic derivatives of these compounds, however may also be a lipase inhibiting polymer. Most preferred are orlistat, panclicins, ATL-962 and lipstatin.

Orlistat (tetrahydrolipstatin) and lipstatin are described in the US-patent U.S. Pat. No. 4,598,089 and its European equivalent EP 0 129 748 B1 in more detail. The compounds are 2-hexyl-3-hydroxy-hexadecanoic acid lactone derivatives with the chemical names (2S,3S,5S,7Z,10Z)-5-((S)-2-formamido-4-methylvaleryloxy)-2-hexyl-3-hydroxy-7,10-hexadecadienoic acid lactone (lipstatin) and (2S,3S,5S)-5-((S)-2-4-methylvaleryloxy)-2-heyl-3-hydroxy-hexadecanoic acid lactone (tetra-hydrolipstatin). The compounds are known to be inhibitors of pancreas lipase which can be used for the prevention of treatment of obesity and hyperlipaemia, for which purpose they can be formulated as medicaments or incorporated into industrially prepared foodstuffs. Inhibition of pancreas lipase prevents the hydrolysis of dietary fats to give absorbable free fatty acids and monoglycerides, so that the fats are excreted unchanged. IC50's for lipstatin and tetrahydrolipstatin for inhibition of hydrolysis of triolein by porcine pancrease lipase are 0.07 and 0.18 mcg/ml, respectively.

Furthermore, there are suitable lipase inhibitors which are structurally related to orlistat and/or lipstatin and which are known as panclicins. These panclicines are derived from orlistat and contain a 4-ring lactone (Mutoh M; Nakada N; Matsukima S; Ohshima S; Yoshinari K; Watanabe J Location: Kanagawa, Japan Issue Date: 19-Jan.-1995 Journal: J. Antibiot., 47, No. 12, 1369-75, 1994). The biological data of these panclicins may be summarized as follows: Panclicins A, B, C, D and E, structural analogs of tetrahydrolipstatin (THL), dose-dependently inhibited hydrolysis of triolein of fatty acids by porcine pancreatic lipase, with IC50 values of 2.9, 2.6, 0.62, 0.66 and 0.89 microM, respectively. The inhibitory activity of panclicins A and B (alanine moiety in place of leucine in THL) was 2-3-fold weaker than that of THL; in contrast, the inhibitory activity of panclicins C, D and E (glycine moiety in place of leucine in THL) was 2-fold stronger than that of THL. Panclicins A, B, C, D and E also potently inhibited plasma lipases with IC50 values of 1.0, 1.2, 0.29, 0.25 and 0,15 microM, respectively. Panclicins A and B inhibited plasma lipases with the same potency as THL, while panclicins C, D and E had a 3-6-fold greater inhibitory activity than THL. Panclicins A, B, C, D and E inhibited bacterial and fungal lipases with profiles similar to those for porcine pancreatic lipase. Panclicins inhibited pancreatic lipase irreversibly, but less irreversibly than THL. Panclicins A, B, C, D and E irreversibly inhibit pancreatic lipase.

Ebelactone B is described in the US-patent U.S. Pat. No. 4,358,602 and its German equivalent DE 3 109 335 C1. Ebelactone A and ebelactone B belong to a group of compounds that exhibit activity to enhance the cell mediated immune response in living animals and they also inhibit inflammations in living animals. Thus they may be used in the immunological treatment of tumours and for enhancing anti-tumour agents such as bleomycins. The compounds have anti-esterase activity and anti-formylmethionine aminopeptidase activity. Administration to mice of these compounds at a dosage of 0.781-50 mg/kg (i.p.) or 0.5 mg/kg (per os) enhances the development of DTH response and the compounds show a potentiating effect on cell-mediated immunity. Ebelactone B reduces carragheenin induced swelling in mice.

In the context of the present invention the lipase inhibitors administered in combination with the CB₁ antagonistic compounds to a patient for treating obesity may be also a polymer that has been substituted with or comprises one or more groups which can inhibit a lipase. Such lipase inhibiting polymers are described in the US patents U.S. Pat. No. 6,572,850, U.S. Pat. No. 6,558,657, U.S. Pat. No. 6,352,692, U.S. Pat. No. 6,267,952 and in the international patent application WO 99/34786. In one embodiment, the lipase inhibiting group can be a “suicide substrate” which inhibits the activity of the lipase by forming a covalent bond with the enzyme either at the active site or elsewhere. In another embodiment, the lipase inhibiting group is an isosteric inhibitor of the enzyme.

In a first aspect of the present invention when using lipase inhibiting polymers in addition to the CB₁ antagonistic compounds, the lipase inhibiting group inactivates a lipase such as gastric, pancreatic and lingual lipases. Inactivation can result by forming a covalent bond such that the enzyme is inactive. The covalent bond can be formed with an amino acid residue at or near the active site of the enzyme, or at a residue which is distant from the active site provided that the formation of the covalent bond results in inhibition of the enzyme activity. Lipases contain a catalytic triad which is responsible for the hydrolysis of lipids into fatty acids. The catalytic triad consists of a serine, aspartate and histidine amino acid residues. This triad is also responsible for the hydrolysis of amide bonds in serine proteases, and it is expected that compounds that are serine protease inhibitors will also inhibit lipases. Therefore, serine protease inhibitors that can be covalently linked to a polymer are preferred lipase inhibiting groups. For example, a covalent bond can be formed between the lipase inhibiting group and a hydroxyl at or the catalytic site of the enzyme. For instance, a covalent bond can be formed with serine. Inactivation can also result from a lipase inhibiting group forming a covalent bond with an amino acid, for example cysteine, which is at some distance from the active site. In a second aspect of the present invention when using lipase inhibiting polymers in addition to the CB₁ antagonistic compounds, non-covalent interaction between the lipase inhibiting group and the enzyme can also result in inactivation of the enzyme. For example, the lipase inhibiting group can be an isostere of a fatty acid, which can interact non-covalently with the catalytic site of the lipase. In addition, the lipase inhibiting group can compete for lipase hydrolysis with natural triglycerides.

A variety of polymers can be employed in the invention described herein. The polymers can be aliphatic, alicyclic or aromatic or synthetic or naturally occurring. However, aliphatic and alicyclic synthetic polymers are preferred. Furthermore, the polymer can be hydrophobic, hydrophilic or copolymers of hydrophobic and/or hydrophilic monomers. The polymer can be non-ionic (e.g., neutral), anionic or cationic, in whole or in part. Furthermore, the polymers can be manufactured from olefinic or ethylenic monomers (such as vinylalcohol) or condensation polymers. For example, the polymers can be a polyvinylalcohol, polyvinylamine, poly-N-alkylvinylamine, polyallylamine, poly-N-alkylallylamine, polyalkylenimine, poly-ethylene, polypropylene, polyether, polyethylene oxide, polyamide, polyacrylic acid, polyalkylacrylate, polyacrylamide, polymethacrylic acid, polyalkylmeth-acrylate, polymethacrylamide, poly-N-alkylacrylamide, poly-N-alkylmethacryl-amide, polystyrene, vinylnaphthalene, ethylvinylbenzene, aminostyrene, vinylbiphenyl, vinylanisole, vinylimidazolyl, vinylpyridinyl, dimethylaminomethyl-styrene, trimethylammoniumethylmethacrylate, trimethylammoniumethylacrylate, carbohydrate, protein and substituted derivatives of the above (e.g., fluorinated monomers thereof) and copolymers thereof. Preferred polymers include polyethers, such as polyalkylene glycols.

The polymers employed in the methods described herein as well as intermediates and methods for preparing the polymers are described in detail in the US patents U.S. Pat. No. 6,572,850, U.S. Pat. No. 6,558,657, U.S. Pat. No. 6,352,692, U.S. Pat. No. 6,267,952 and in the international patent application WO 99/34786, which are all incorporated by reference into the present invention.

Recently, in the international patent application WO 03/072555 new 5-hydrocarbyloxy-3-phenyl-1,3,4-oxadiazol-2-ones of formula (A) are described to be pancreatic lipase inhibitors useful for treating metabolic diseases, cardiovascular diseases or especially obesity.

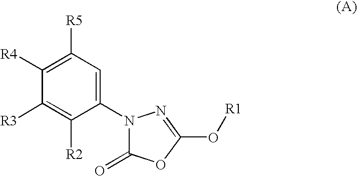

Such oxadiazolones of formula (A) and their salts and acid addition salts are also suitable for combinations with the CB₁ antagonistic compounds used according to the present invention. In formula (A) the substituents may be as follows:
R1 may be 7-22C alkyl; 2-4C alkyl substituted by 4-20C alkoxy, 6-10C aryl, 6-10C aryloxy or (4-12C) alkoxy-(2-4C) alkoxy (where aryl can be substituted by one or more of halogen, 14C alkyl, 1-4C alkoxy, NO2 or CF3); 7-20C alkenyl; or phenyl substituted by 6-12C alkyl or by phenoxy; and
R2 to R5 each may be H, halogen, NO2, 1-4C alkyl, 1-4C alkoxy, CF3 or OCF3; or (6-10C) aryl-(1-4C) alkoxy, 6-10C aryloxy, 6-10C aryl, 3-8C cycloalkyl or 3-8C cycloalkoxy (optionally substituted by halogen, CF3, 1-4C alkoxy or 1-4C alkyl).

These 5-hydrocarbyloxy-3-phenyl-1,3,4-oxadiazol-2-ones are described to have pharmacological properties as anorectic, antidiabetic, hypotensive or cardiant, with mechanism of action as pancreatic lipase inhibitors. For example 5-dodecyloxy-3-(4-trifluoromethoxy-phenyl)-3H-(1,3,4)-oxadiazol-2-one had IC50 0.03 microM for inhibition of porcine pancreatic lipase. Hence, these 5-hydrocarbyloxy-3-phenyl-1,3,4-oxadiazol-2-ones may be used as medicaments, especially for the treatment of obesity. As pancreatic lipase inhibitors, the 5-hydrocarbyloxy-3-phenyl-1,3,4-oxadiazol-2-ones inhibit the resorption of the fat content of foods and thus reduce fat uptake and body weight (or prevent increase in body weight). Furthermore, the 5-hydrocarbyloxy-3-phenyl-1,3,4-oxadiazol-2-ones are reported to also have a beneficial effect in the treatment of metabolic disorders (e.g. diabetes) or cardiovascular disorders (e.g. hypertension and cardiac infarction). The lipase inhibiting compounds of formula (A) are described in more detail in the WO 03/072555 and can be obtained according to known methods. A suitable synthesis for the lipase inhibiting compounds of formula (A) is described also in the international patent application WO 03/072555. The whole content of the international patent application WO 03/072555 is incorporated by reference into the present application regarding the disclosure of lipase inhibitors of formula (A).

In addition, in the international patent application WO 03/072098 further 5-hydrocarbyloxy-3-phenyl-1,3,4-oxadiazol-2-ones of formula (A) are described to be pancreatic lipase inhibitors useful for treating of obesity or diabetes mellitus type 1 and 2. Such oxadiazolones of formula (A) as described in WO 03/072098 and their salts and acid addition salts are also suitable for combinations with the CB₁ antagonistic compounds used according to the present invention. In formula (A) the substituents may be as follows:
R1 may be 1-6C alkyl; 3-9C-cycloalkyl, both groups optionally may be substituted by phenyl, 1-4C alkoxy, S-1-4C alkyl, N(1-4C-alkyl)₂; and phenyl optionally may be also substituted by halogen, 14C alky, 14C-alkyloxy, nitro or CF3; and
R2 to R5 each may be independently H, halogen, NO2, 1-4C alkyl, 1-9C alkoxy which is substituted by F, 6-10C-aryl, amino or 1-4C alkyl amino; 6-10C-aryl-1-4C-alkyloxy, 6-10C-aryloxy, 6-10C-aryl, 6-10C-aryloxy-1-4C-alkyl, 3-8C cycloalkyl or O(3-8 cycloalky), which may optionally be substituted by halogen, CF3, 1-4 alkyloxy or 1-4C alkyl;
   SO2-NH-(1-6C alkyl), optionally substituted by N(1-6C alkyl)2, SO2-NH-(2,2,6,6-tetramethylpiperidin-4-yl), SO2-NH-(3-8C cycloalkyl), optionally substituted by 1-4C alkyl, SO2—N(1-6Calkyl)₂ or COX;
   2-oxo-pyrrolidin-1-yl, 2,5-dimethylpyrrol-1-yl or NR6-A-R7
   with the proviso that R2, R3, R4 and R5 are not H at the same time when
   X is O(1-6C alkyl), NH(1-6C alkyl), NH(3-8C cycloalkyl or N(1-6C alkyl)2 and N(1-6C alkyl)2 may be also pyrrolidino, piperidino, morpholino, thiomorpholino or piperazino, which optionally substituted by 1-4C alkyl, benzyl, 6-1° C. aryl, CO-(14 alkyl), CO-(6-10 aryl), CO—O—(1-4C alkyl), SO2-(1-4C alkyl) or SO2-(6-10C aryl);
   R6 is H, 1-4C alkyl or 6-10C-aryl-1-4C-alkyl, wherein aryl may be substituted by halogen, CF3, 1-8C alkyloxy or 1-4C alkyl;
   A is a single bond, COn, SOn or CONH;
   n is 1 oder2:
   R7 is H; 1-18C alkyl or 2-18C alkenyl, which may up to three times substituted by 1-4C alkyl, halogen, CF3, 1-4C alkyloxy, N(1-4C alkyl)2, —COOH, 1-4C alkyloxycarbonyl, 6-12C aryl, 6-12C aryloxy, 6-12C arylcarbonyl, 6-10-aryl-1-4C-alkyloxy or oxo, wherein aryl itself may be optionally substituted by halogen, 1-4C alkyl, aminosulfonyl or methylmercapto;
      6-10C-aryl-1-4C-alkyl, 5-8C-cycloalkyl-1-4C-alkyl, 5-8C cycloalkyl, 6-10-aryl-2-6C-alkenyl, 6-1° C. aryl, biphenylyl, diphenyl-(1-4 alkyl), indanyl, which may be optionally substituted by 1-18C alkyl, 1-18C alkyloxy, 3-8C cycloalkyl, COOH, hydroxy, 1-4C alkylcarbonyl, 6-10C-aryl-1-4C alkyl, 6-10C-aryl-1-4C-alkyloxy, 6-1° C. aryloxy, nitro, cyano, 6-1° C. aryl, fluorsulfonyl, 1-6C alkyloxycarbonyl, 6-10 arylsulfonyloxy, pyridyl, NHSO-2-(6-10 aryl), halogen, CF3 or OCF3, wherein alkyl may be additionally substituted by 1-4C alkyloxycarbonyl, CF3 or carboxy and aryl by halogen, CF3 or 1-4C alkyloxy;
      or the group Het-(CH2)r- with r=0, 1, 2 or 3 and Het=saturated or unsaturated 5-7-membered heterocyclus, which may be optionally benzo anellated and substituted by 1-4C alkyl, 6-10C aryl, halogen, 1-4C alkyloxy, 1-4C alkyloxycarbonyl, 6-10C-aryl-1-4Calkyl, 6-10C-aryl-1-4C-alkylmercapto or nitro, wherein benzo anellated aryl may be substituted by halogen, 1-4C alkyloxy or CF3 and alkyl in arylalkyl may be substituted by methoxy and CF3.

The lipase inhibiting compounds of formula (A) are described in more detail in the WO 03/072098 and can be obtained according to known methods. A suitable synthesis for the lipase inhibiting compounds of formula (A) is described also in the international patent application WO 03/072098. The whole content of the international patent application WO 03/072098 is incorporated by reference into the present application regarding the disclosure of lipase inhibitors of formula (A).

In addition, in the US patent U.S. Pat. No. 6,624,161 and its corresponding international patent application WO 00/040,569 and WO 00/40247 further lipase inhibiting compounds are described which are also suitable in the context of the present invention for combination with CB₁ antagonistic compounds described herein. These patent documents U.S. Pat. No. 6,624,161 and WO 00/040,569 describe a series of compounds which are 2-oxy-4H-3,1-benzoxazin-4-one derivatives, including ATL-962, and their use in obesity and obesity-related disorders, including type 2 diabetes. The 2-oxy-4H-3,1-benzoxazin-4-one derivatives have the formula (B) or are or a pharmaceutically acceptable salt, ester, amide or prodrug thereof: 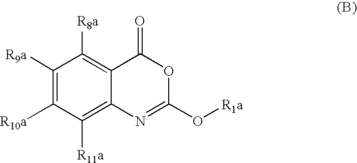
wherein:
R1a is
   (i) a C10-30 branched or unbranched alkyl, optionally substituted by one or more independently of C3-6 cycloalkyl, C3-6 cycloalkenyl, aryl, heteroaryl, reduced heteroaryl, —C(O)R13, —CO2R13, —SOR13, —SO2R13, —NR13R14, —OR13,
      —SR13, —C(O)NR13R14, —NR14C(O)R13, halogen, cyano, and nitro and/or optionally interrupted by one or more oxygen atoms with the proviso that any hetero atom in R1a must be separated from the exocyclic oxygen atom (or from any other heteroatom) by at least two carbon atoms;
   (ii) C2-25 alkenyl, C2-25 alkynyl, C3-6 cycloalkenyl, aryl-C2-25 alkenyl, heteroaryl-C2-25 alkenyl, reduced heteroaryl, reduced heteroaryl-C1-25 alkyl or a substituted derivative of any of the foregoing groups wherein the substituents are one or more independently of C1-6 alkyl, halosubstituted C1-6 alkyl, aryl, aryl-C1-6 alkyl, heteroaryl, reduced heteroaryl, reduced heteroaryl-C1-6 alkyl, C1-6 alkoxy, aryl-C1-6 alkoxy, —C(O)R13, —CO2R13, —SOR13, —SO2R13, —NR13R14, —OR13, —SR13, —C(O)NR13R14, —NR14C(O)R13, halogen, cyano, and nitro, with the proviso that any hetero atom in R1a must be separated from the exocyclic oxygen atom (or from any other heteroatom) by at least two carbon atoms;
   (iii) a C2-9 alkyl group interrupted by one or more oxygen atoms and optionally substituted by one or more independently of C3-6 cycloalkyl, C3-6 cycloalkenyl, aryl, heteroaryl, reduced heteroaryl, —C(O)R13, —CO2R13, —SOR13, —SO2R13, NR13R14, OR13, SR13, —C(O)NR13R14, —NR14C(O)R13, halogen, cyano and nitro with the proviso that any hetero atom in R1a must be separated from the exocyclic oxygen atom (or from any other heteroatom) by at least two carbon atoms; or
   (iv) a C1-9 alkyl group substituted by a group selected from —C(O)R13, —CO2R13, SOR13, SO2R13, NR13R14, OR13, SR13, C(O)NR13R14, NR14C(O)R13; tetrahydronaphthyl, pyridyl, pyrrolyl, piperidinyl, halogen, cyano, nitro, bicyclic aryl, bicyclic heteroaryl, monocyclic or bicyclic reduced heteroaryl, monocyclic heteroaryl other than imidazolyl;
   (v) a phenyl group substituted by a group selected from OR17, —COR13, —CO2R13, SOR13, SO2R13, CONR13R14, NR14C(O)R13; halosubstituted C1-6 alkyl, aryl, arylC1-6 alkyl, heteroaryl and heteroarylC1-6 alkyl; or
   (vi) a bicyclic aryl, bicyclic heteroaryl, monocyclic or bicyclic reduced heteroaryl, or monocyclic heteroaryl group other than imidazolyl, optionally substituted by a group selected from OR17, —COR13, —CO2R13, SOR13, SO2R13, CONR13R14,
      NR14 C(O)R13; halosubstituted C1-6 alkyl, aryl, arylC1-6 alkyl, heteroaryl and heteroarylC1-6 alkyl;
      where R13 and R14 each independently represents hydrogen, C1-10 alkyl, C2-10 alkenyl, C2-10 alkynyl, C3-6 cycloalkyl, C3-6 cycloalkenyl, aryl, arylC1-10 alkyl, heteroaryl, heteroarylC1-10 alkyl, reduced heteroaryl or reduced heteroaryl,
      C1-10 alkyl, and R17 represents hydrogen or C2-10 alkenyl, C2-10 alkynyl, C3-6 cycloalkyl, C3-6 cycloalkenyl, aryl, arylC1-10 alkyl, heteroaryl, heteroarylC1-10 alkyl, reduced heteroaryl or reduced heteroarylC1-10 alkyl
and R8a, R9a, R10a and R11a
   are each independently hydrogen, halo, hydroxy, amino, nitro, cyano, thiol, C1-10 alkyl,
C1-10 alkoxy, C1-10 cycloalkyl, C1-10 cycloalkoxy, C(O)R15, C(O)NR15R16, S(O)R15 or haloC1-10 alkyl;
where R15 and R16 each independently represent hydrogen or C1-10 alkyl with the proviso that when R8a, R9a, R10a, and R11a are H, R1a is not CH2CH2C1 or C3 alkenyl.

Furthermore in the international patent application WO 00/40247 related 2-amino-4H-3,1-benzoxazin-4-one derivatives are described as lipase inhibiting compounds for the treatment of obesity. In formula (B) then the —OR1a substituent is replaced by a —NR1 R2 group with the definitions for R1 and R2 as given in the WO 00/40247.

The above group of structurally related compounds include ATL-962, an oral non-absorbed synthetic lipase inhibitor derived from Alizyme's pancreatic lipase inhibitor research program, is under development for the potential treatment of obesity and the potential management of type 2 diabetes. ATL-962 has the chemical name 2-hexadecyloxy-6-methyl-4H-3,1-benzoxazin-4-one. Preclinical studies showed that ATL-962 had similar efficacy to orlistat and no toxicity was observed. Clinical data for these compounds is also available in the public domain, e.g. resulting from clinical studies with ATL-962 in obesity.

Thus, the results from a phase lb program with ATL-962 were presented at the International Congress of Obesity in Sao Paulo, Brazil. The three phase lb trials involved a total of 99 healthy male volunteers in groups of seven or nine, given one of several doses of ATL-962 (66 subjects) or placebo (24 subjects), tid with food for 5 days. In one group the nine subjects were given orlistat (qv) 120 mg tid. Overall, ATL-962 was safe and well tolerated and showed evidence of efficacy as indicated by an increase in excretion of fat from the diet. Subjects given doses between 50 mg and 300 mg ATL-962 bid with meals excreted fat at an average of between 4.9 (+/−4.3) and 11.2 (+/−6.9) g/day compared to 1.4 (+/−1.0) g/day on placebo and 5.6 (+/−3.8) g/day on orlistat. Compared to placebo, 55% of subjects who received ATL-962 (50 mg to 300 mg) demonstrated a 3-fold or greater increase in fat excretion and 27% of subjects demonstrated a 7-fold or greater increase. There was evidence of dose-dependency. Adverse events and their frequency were similar between ATL-962 and placebo and were mainly gastrointestinal, with the predominant event being oily stool.

The results of a multicenter, randomized, double-blind, parallel-group trial (phase IIb study), involving 370 clinically obese patients, was being performed in specialist clinics in 5 European countries, and in September 2003 preliminary results were reported. All dose levels of ATL-962 (60, 120 and 240 mg) demonstrated a significant reduction in weight, compared to placebo, for all treatment groups. There was no difference in the extent of weight loss between treatment groups. LDL-cholesterol decreased in the treatment groups, but not for placebo. There was no difference in HDL-cholesterol levels in the treatment groups, whilst it increased in placebo-treated patients. Total cholesterol decreased in the treatment groups, whilst placebo showed an increase. ATL-962 was safe and generally well tolerated.

The lipase inhibiting compounds of formula (B) like ATL-962 and structurally related compounds are described in more detail in the US patent U.S. Pat. No. 6,624,161 and its corresponding international patent application WO 00/040,569, and can be obtained according to known methods. A suitable synthesis for the lipase inhibiting compounds of formula (B) is described also in the U.S. Pat. No. 6,624,161 and international patent application WO 00/040,569. The whole content of the U.S. Pat. No. 6,624,161 and international patent application WO 00/040,569 is incorporated by reference into the present application regarding the disclosure of lipase inhibitors of formula (B). The whole content of the international patent application WO 00/040,247 is also incorporated by reference into the present application regarding the disclosure of lipase inhibitors described therein, with related 2-amino-4H-3,1-benzoxazin-4-one compound structure.

Pharmaceutically acceptable salts, hydrates and solvates, and prodrugs of all the above described lipase inhibiting compounds may also be used in the context of the present invention.

The CB₁ antagonistic compound, in particular a 4,5-dihydro-1H-pyrazole derivative which is a potent and selective antagonist of the cannabis CB₁-receptor, preferably having of formula (I), or a prodrug, tautomer or salt thereof, and the lipase inhibiting compound used according to the invention can be brought into forms suitable for treatment and/or prophylaxis of obesity, e.g. for adolescent or pediatric administration, as well as for the administration in treating drug induced obesity by means of usual processes using pharmaceutical excipients, auxiliary substances and/or liquid or solid carrier materials. As therapeutic agents, the CB₁ antagonistic compound and/or the lipase inhibiting compounds may be contained together with (conventional) pharmaceutical excipients, adjuvants and/or auxiliaries in pharmaceutical preparations such as tablets, capsules, suppositories or solutions. These pharmaceutical preparations may be prepared according to known methods, using conventional solid or liquid vehicles such as lactose, starch or talc, or liquid paraffins and/or using (conventional) pharmaceutical excipients, adjuvants and/or auxiliaries, such as tablet disintegrating agents, solubilisers or preservatives.

Hence, in a further aspect the invention also pertains to a pharmaceutical composition containing at least one CB₁ antagonistic compound, in particular a 4,5-dihydro-1H-pyrazole derivative which is a potent and selective antagonist of the cannabis CB₁-receptor, or a prodrug, tautomer or salt thereof, in combination with at least one lipase inhibiting compound. A preferred pharmaceutical composition contains at least one compound of formula (I) as defined above in combination with at least one lipase inhibiting compound as combined active components. A further pharmaceutical composition according to the invention contains as active components at least one CB₁ antagonistic compound, preferably the CB₁ antagonistic having formula (I) as defined above, or a prodrug, tautomer or salt thereof, and at least one lipase inhibiting compound for the treatment and/or prophylaxis of obesity in adolescent or in juvenile patients and/or for the treatment and/or prophylaxis of drug induced obesity in juvenile as well as adolescent patients. Particular pharmaceutical compositions according to the invention, are characterized in that the at least one CB₁ antagonistic compound, preferably the CB₁ antagonistic having formula (I) as defined above, or the prodrug, tautomer or salt thereof, and the at least one lipase inhibiting compound each are present in an amount effectively suited for the treatment and/or prophylaxis of obesity in a juvenile patient in need of such treating. In a further embodiment of the invention the CB₁ antagonistic compound, in particular the CB₁ antagonistic compound of formula (I), and the lipase inhibiting compound are each present in the pharmaceutical composition in an amount effectively suited for the treatment and/or prophylaxis of drug induced obesity in juvenile as well as adolescent patients in need of such treating. In the pharmaceutical compositions according to the invention the CB₁ antagonistic compound, preferably the CB₁ antagonistic having formula (I), or the prodrug, tautomer or salt thereof, is used preferably in combination with at least one lipase inhibiting compound selected from the group of lipase inhibiting polymers, orlistat, panclicins, ATL-962 and lipstatin.

The invention also pertains to a pharmaceutical product containing as a medicament a CB₁ antagonistic compound, preferably the CB₁ antagonistic compound having formula (I) as defined in claim 2, or a prodrug, tautomer or salt thereof, and a leaflet indicating that said CB₁ antagonistic compound may be administered in combination with a lipase inhibiting compound for simultaneous, separate or step-wise administration in the treatment and/or prophylaxis of obesity.

Finally the invention also includes a method of treatment and/or prophylaxis of obesity, e.g. in adolescent or in juvenile patients and/or for the treatment and/or prophylaxis of drug induced obesity in juvenile as well as adolescent patients, characterized in that a CB₁ antagonistic compound, in particular a 4,5-dihydro-1H-pyrazole derivative which is a potent and selective antagonist of the cannabis CB₁-receptor, or a prodrug, tautomer or salt thereof, is administered in combination with at least one lipase inhibiting compound to a patient in need of such treating. In a preferred method of treatment and/or prophylaxis of obesity according to the invention a CB₁ antagonistic compound which is a 4,5-dihydro-1H-pyrazole derivative of formula (I) as defined above, or a prodrug, tautomer or salt thereof, is administered in combination with at least one lipase inhibiting compound. The method of treatment and/or prophylaxis of obesity according to the present invention may be directed to obesity in adolescent or in juvenile patients and/or to drug induced obesity in juvenile as well as adolescent patients. In a variant of the invention the method of treatment and/or prophylaxis is characterized in that the treating is directed to obesity in juvenile patients. In a further variant of the invention the method of treatment and/or prophylaxis is characterized in that the treating is directed to drug induced obesity in juvenile or adolescent patients. In the method of treatment and/or prophylaxis according to the invention the CB₁ antagonistic compound, preferably the CB₁ antagonistic compound having formula (I) as defined above, or a prodrug, tautomer or salt thereof, is administered preferably in combination with at least one lipase inhibiting compound selected from the group of lipase inhibiting polymers, orlistat, panclicins, ATL-962 and lipstatin.

According to the invention the CB₁ antagonistic compound, preferably the CB₁ antagonistic compound having formula (I) as defined above, or a prodrug, tautomer or salt thereof, is administered in combination with the lipase inhibiting compound by simultaneous, separate or step-wise administration route.

The compounds used in the combinations or compositions according to the present invention each are preferably administered to a patient in need thereof and in a quantity sufficient to prevent and/or treat the symptoms of the condition, disorder or disease, e.g. obesity. For all aspects of the invention, particularly medical ones, the administration of a compound or composition has a dosage regime which will ultimately be determined by the attending physician and will take into consideration such factors such as the compound being used, animal type, age, weight, severity of symptoms, method of administration, adverse reactions and/or other contraindications. Specific defined dosage ranges can be determined by standard design clinical trials with patient progress and recovery being fully monitored. Such trials may use an escalating dose design using a low percentage of the maximum tolerated dose in animals as the starting dose in man.

The physiologically acceptable compounds used in the combinations or compositions according to the present invention each are will normally be administered in a daily dosage regimen (for an adult patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a physiologically acceptable salt thereof calculated as the free base, the compound being administered 1 to 4 times per day. Suitably the compounds will be administered for a period of continuous therapy, for example for a week or more. For a juvenile patient usually a part of the oral dose for an adult patient is administered, e.g. 1 fifth to 1 half of the oral dose described before for an adult patient.

Preferably, in one embodiment of the invention the method of treatment and/or prophylaxis is directed to the treating of obesity in juvenile patients. In another preferred embodiment of the invention the method of treatment and/or prophylaxis is directed to the treating of drug induced obesity in juvenile or adolescent patients. This drug induced obesity may be in particular caused by drugs like atypical antipsychotics.

In one embodiment of the invention the method of treatment and/or prophylaxis is directed to the treating of obesity in juvenile patients. Thus, it is advantageous that Cannabinoid antagonists in combination with lipase inhibitors are particularly suitable for the treatment of Childhood Obesity and related Comorbidities as for example Type 2 Diabetes. There is a clear medical need for improved therapy as obesity has become an increasingly important medical problem not only in the adult population but increasingly in children and (young and older) adolescents. In national surveys from the 1960s to the 1990s in the United States, the prevalence of overweight in children grew from 5% to 11% (Sorof and Daniels 2002). In Canada as another example childhood obesity has tripled in the past 20 years (Spurgeon 2002). Obesity in childhood causes a wide range of serious complications, and increases the risk of premature illness and death later in life, raising public-health concerns (Ebbeling, Pawlak et al. 2002). Over the last decades a tremendous increase of cases of type 2 diabetes was observed, especially also in children. This epidemic trend is clearly reflecting the increasing rates of obesity. Type-2-diabetes was in the past considered a disease of adults and older individuals, not a pediatric condition (Arslanian 2002). One of the main risk factor of pediatric type 2 diabetes is obesity.

Type 2 diabetes in children (as is in adults) is part of the insulin resistance syndrome (Rosenbloom 2002) that includes hypertension, dyslipidemia and other atherosclerosis risk factors, and hyperandrogenism seen as premature adrenarche and polycystic ovary syndrome. Other outcomes related to childhood obesity include left ventricular hypertrophy, nonalcoholic steatohepatitis, obstructive sleep apnea, orthopedic problems, and severe psychosocial problems.

In addition primary hypertension has become increasingly common in children again associated obesity as a major independent risk factor. Obese children are at approximately a 3-fold higher risk for hypertension than non-obese children (Sorof and Daniels 2002). The benefits of weight loss for blood pressure reduction in children have been demonstrated in both observational and interventional studies.

Public concerns are rising because of a rapid development of the childhood obesity epidemic in genetically stable populations. Driving factors are assumed to be mainly adverse environmental factors for which straightforward recommendations of life style modifications exists. Obesity and it's related co-morbidities are very serious medical conditions and state of the art measures and treatment of obesity and especially childhood obesity remain largely ineffective at the time being (Ebbeling, Pawlak et al. 2002). The management of type 2 diabetes in is also especially difficult in children and the adolescent age group (Silink 2002). Craving for and over consumption of palatable food is one of the important factors of life-style related obesity in humans and especially also in children and adolescents. Treatment of type 2 diabetes and other co-morbid conditions by the degree of metabolic derangement and symptoms: The only data on the use of oral hypoglycemic agents in children with type 2 diabetes has been with metformin (Rosenbloom 2002).

Thus, CB₁ antagonists used according to the present invention in combination with lipase inhibitors offer a unique opportunity for the treatment of obesity by interacting with these “driving forces”. They are superior to current medical treatments and especially suited for adolescent as well as pediatric treatment because of their outstanding safety profile and/or tolerability and surprisingly beneficial combination effects. Besides efficacy, the treatment of obesity, especially the treatment of childhood obesity, dictated by safety.

Obesity in childhood is a medical condition that is likely to require long-term management. The safety profile of CB₁ antagonists according to the present invention in combination with lipase inhibitors are suggested to be superior to current standard medications, and these CB₁ antagonists in combination with lipase inhibitors will be especially suited for the treatment and prevention of obesity in adolescents and in childhood obesity and related co-morbidities.

### LITERATURE

Arslanian, S. (2002). “Type 2 diabetes in children: clinical aspects and risk factors.” Horm Res 57 Suppl 1: 19-28.
Ebbeling, C. B., D. B. Pawlak, et al. (2002). “Childhood obesity: public-health crisis, common sense cure.” Lancet 360(9331): 473-82.
Rosenbloom, A. L. (2002). “Increasing incidence of type 2 diabetes in children and adolescents: treatment considerations.” Paediatr Drugs 4(4): 209-21.
Silink, M. (2002). “Childhood diabetes: a global perspective.” Horm Res 57 Suppl 1:1-5.
Sorof, J. and S. Daniels (2002). “Obesity hypertension in children: a problem of epidemic proportions.” Hypertension 40(4): 441-7.
Spurgeon, D. (2002). “Childhood obesity in Canada has tripled in past 20 years.” Bmj 324(7351): 1416.

In another embodiment of the invention the method of treatment and/or prophylaxis is directed to the treating of drug induced obesity in juvenile or adolescent patients. Drug induced weight gain is also of major concern and subject to high medical need of improved treatments. Again, in this context the CB₁ antagonists in combination with lipase inhibitors according to the present invention are suggested to be superior to current standard medications, and these CB₁ antagonists in combination with lipase inhibitors will be especially suited for the treatment and prevention of drug induced obesity in juvenile as well as in adolescent patients.

Regarding drug induced weight gain, it is reported by Zimmermann, U., T. Kraus, et al. (2003, “Epidemiology, implications and mechanisms underlying drug-induced weight gain in psychiatric patients.” J Psychiatr Res 37(3): 193-220) that body weight gain frequently occurs during drug treatment of psychiatric disorders and is often accompanied by increased appetite or food craving. While occurrence and time course of this side effect are difficult to predict, it ultimately results in obesity and the morbidity associated therewith in a substantial part of patients, often causing them to discontinue treatment even if it is effective. Weight gain appears to be most prominent in patients treated with some of the second generation antipsychotic drugs and with some mood stabilizers. Marked weight gain also frequently occurs during treatment with most tricyclic antidepressants.

Very large weight gains are associated with drugs like for example the atypical antipsychotics clozapine and olanzapine. Some atypical antipsychotics, however, tend to cause significant weight gain, which may lead to poor compliance and other adverse health effects (Nasrallah, H. (2003). “A review of the effect of atypical antipsychotics on weight.” Psychoneuroendocrinology 28 Suppl 1: 83-96.). The mechanisms involved in antipsychotic drug-related weight gain are as yet uncertain, although serotoninergic, histaminic, and adrenergic affinities have been implicated along with other metabolic mechanisms. The atypical antipsychotics vary in their propensity to cause weight change with long-term treatment. Follow-up studies show that the largest weight gains are associated with clozapine and olanzapine, and the smallest with quetiapine and ziprasidone. Risperidone is associated with modest weight changes that are not dose related. Given the equivalent efficacy of atypical antipsychotics, weight-gain profile is a legitimate factor to consider when constructing an algorithm for treatment due to the serious medical consequences of obesity. In this regard co-administration of a CB₁ antagonist in combination with lipase inhibitors according to the invention is suggested to work beneficially.

### Experimental Protocol for Study of CB₁ Antagonistic Compound Plus Lipase Inhibitor Effects on Obesity

The beneficial pharmacological effects of the combination of a CB₁ antagonist with a lipase inhibitor according to the invention can be shown by standard experimental animal models by measuring the influence of the administered combination of a CB₁ antagonist with a lipase inhibitor on the driving and characteristic parameters associated with obesity.

For investigation of the influence of the combination of a CB₁ antagonist with a lipase inhibitor on obesity the body weight gain in rats may be measured as a pharmacological indicator. Here fore, the following experimental protocol for rats may be applied:

The rats will have unlimited access to feed for two 2.5 h periods per day, during the dark phase of a reversed 12 h/12 h light cycle, e.g. lights are put on at 21.15 h and put off at 09.15 h. The rats will be offered a high fat, high sucrose diet (Western diet). The lipase inhibitor will be dosed immediately before the rats are fed. The CB₁ antagonist will be dosed 1 h before the lipase inhibitor is administered.

As an example, the following daily dosing schedule is applicable for a given period of e.g. days, weeks or months:

The CB₁ antagonist, in particular the CB₁ antagonisic compound of formula (I) as defined above, or a vehicle dose is administered (po) in the morning at ca. 07.45 to 08.00 h. The lipase inhibitor, e.g. in particular orlistat, or a vehicle dose is administered (po) ca. 08.45 to 09.00 h. After medication the rats are set to ad-libitum feed from 09.15 to 11.45 h, followed by feed removal from about 11.45 to 14.45 h. Another dose of lipase inhibitor, e.g. in particular orlistat, or vehicle (Labrasol) dose is administered (po) in the afternoon at about 14.15 to 14.30 h, followed by ad-libitum feed from 14.45 to 17.15 h. Thereafter, the rats are set to feed deprivation from 17.15-09.15 h.

The experimental protocol results will compare daily food intake and body weight gain as indicators for the effects of the combination treatment on obesity during the experimental phase. In addition to the before given parameters it may be desired to also collect faeces and to estimate fat digestibility. Eventually it may be also desired to perform a carcass analysis.

Furthermore, after finishing the experimental feeding and administering phase, biochemical parameters may be measured at slaughter of the rats.

For investigating the effects the total number of rats subject to the experimental protocol is divided into the following groups with approximately the same number of rats in each group:
1) Control group: The rats receive only vehicle according to the protocol to simulate administration (placebo group).
2) CB₁ group: The rats receive a CB₁ antagonist in a vehicle.
3) LI group: The rats receive as lipase inhibitor (“LI”) e.g. the compound orlistat in a vehicle.
4) CB₁+LI group (combination group): The rats receive a CB₁ antagonist in a vehicle and as lipase inhibitor (“LI”) e.g. the compound orlistat in a vehicle.

The results of this protocol and the respective investigations show the beneficial suitability of the combination of a CB₁ antagonist and a lipase inhibitor in the treatment and/or prophylaxis of obesity.

1. Use of a CB₁ antagonistic compound, in particular a 4,5-dihydro-1H-pyrazole derivative which is a potent and selective antagonist of the cannabis CB₁-receptor, or a prodrug, tautomer or salt thereof, in combination with at least one lipase inhibiting compound in the manufacture of a medicaments for the treatment and/or prophylaxis of obesity, including in particular the treatment and/or prophylaxis of obesity in juvenile patients and/or drug induced obesity in juvenile as well as adolescent patients.

2. Use according to claim 1 of a CB₁ receptor antagonistic compound of formula (I), a prodrug thereof, a tautomer thereof or a salts thereof, in combination with at least one lipase inhibiting compound, in the manufacture of a medicament for the treatment and/or prophylaxis of obesity in adolescent or in juvenile patients and/or for the treatment and/or prophylaxis of drug induced obesity in juvenile as well as in adolescent patients: 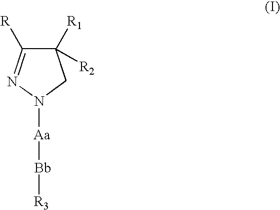
wherein
R and R₁ are the same or different and represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
Aa represents one of the groups (i), (ii), (iii), (iv) or (v) 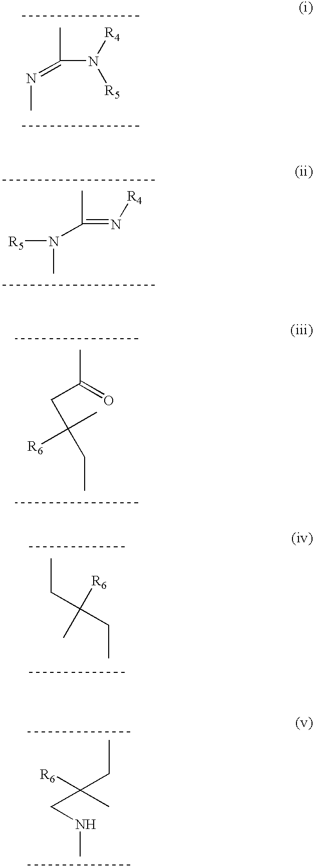
wherein
R₄ and R₅ independently of each other represent hydrogen or C₁₋₈ branched or unbranched alkyl or C₃₋₈ cycloalkyl or R₄ represents acetamido or dimethylamino or 2,2,2-trifluoroethyl or phenyl or pyridyl with the proviso that R₅ represents hydrogen
R₆ represents hydrogen or C₁₋₃ unbranched alkyl
Bb represents sulfonyl or carbonyl,
R₃ represents benzyl, phenyl, thienyl or pyridyl which may be substituted with 1, 2 or 3 substituents Y, which can be the same or different, or R₃ represents C₁₋₈ branched or unbranched alkyl or C₃₋₈ cycloalkyl, or R₃ represents naphtyl.

3. Use of the compound having formula (I) according to claim 2, wherein R is the group 4-chlorophenyl, R₁ is phenyl, R₂ is hydrogen, Aa is the group (i) wherein R₄ is hydrogen and R₅ is methyl, Bb is sulfonyl, and R₃ represents 4-chlorophenyl, and salts thereof.

4. Use of the compound having formula (I) according to claim 2, wherein the compound is a levorotatory enantiomer.

5. Use of the compound having formula (I) according to anyone of the claims 1 to 4, wherein the CB₁ antagonistic compound is used in combination with at least one lipase inhibiting compound selected from the group of lipase inhibiting polymers, orlistat, panclicins, ATL-962 and lipstatin.

6. A pharmaceutical composition containing at least one CB₁ antagonistic compound, in particular a 4,5-dihydro-1H-pyrazole derivative which is a potent and selective antagonist of the cannabis CB₁-receptor, or a prodrug, tautomer or salt thereof, in combination with at least one lipase inhibiting compound.

7. A pharmaceutical composition according to claim 6 containing at least one CB₁ antagonistic compound of formula (I) as defined in claim 2, or a prodrug, tautomer or salt thereof, in combination with at least one lipase inhibiting compound.

8. A pharmaceutical composition according to any of the claims 6 to 7 containing as active components at least one CB₁ antagonistic compound, preferably the CB₁ antagonistic having formula (I) as defined in claim 2, or a prodrug, tautomer or salt thereof, and at least one lipase inhibiting compound for the treatment and/or prophylaxis of obesity in adolescent or in juvenile patients and/or for the treatment and/or prophylaxis of drug induced obesity in juvenile as well as adolescent patients.

9. A pharmaceutical composition according to any of the claims 6 to 8, wherein the at least one CB₁ antagonistic compound, preferably the CB₁ antagonistic having formula (I) as defined in claim 2, or the prodrug, tautomer or salt thereof, and the at least one lipase inhibiting compound each are present in an amount effectively suited for the treatment and/or prophylaxis of obesity in a juvenile patient in need of such treating.

10. A pharmaceutical composition according to any of the claims 6 to 8, wherein the at least one CB₁ antagonistic compound, preferably the CB₁ antagonistic having formula (I) as defined in claim 2, or the prodrug, tautomer or salt thereof, and the at least one lipase inhibiting compound each are present in an amount effectively suited for the treatment and/or prophylaxis of drug induced obesity in juvenile as well as adolescent patients in need of such treating.

11. A pharmaceutical composition according to any of the claims 6 to 10, wherein the CB₁ antagonistic compound, preferably the CB₁ antagonistic having formula (I) as defined in claim 2, or the prodrug, tautomer or salt thereof, is used in combination with at least one lipase inhibiting compound selected from the group of lipase inhibiting polymers, orlistat, panclicins, ATL-962 and lipstatin.

12. A method of treatment and/or prophylaxis of obesity, characterized in that a CB₁ antagonistic compound, in particular a 4,5-dihydro-1H-pyrazole derivative which is a potent and selective antagonist of the cannabis CB₁-receptor, or a prodrug, tautomer or salt thereof, is administered in combination with at least one lipase inhibiting compound to a patient in need of such treating.

13. A method of treatment and/or prophylaxis of obesity according to claim 12, characterized in that a CB₁ antagonistic compound as defined in claim 2, or a prodrug, tautomer or salt thereof, is administered in combination with at least one lipase inhibiting compound.

14. A method of treatment and/or prophylaxis of obesity according to any of the claims 12 to 13, characterized in that the treatment and/or prophylaxis is directed to obesity in adolescent or in juvenile patients and/or to drug induced obesity in juvenile as well as adolescent patients.

15. A method of treatment and/or prophylaxis according to any of the claims 12 to 14, characterized in that the treating is directed to obesity in juvenile patients.

16. A method of treatment and/or prophylaxis according to any of the claims 12 to 14, characterized in that the treating is directed to drug induced obesity in juvenile or adolescent patients.

17. A method of treatment and/or prophylaxis according to any of the claims 12 to 16, characterized in that the CB₁ antagonistic compound, preferably the CB₁ antagonistic compound having formula (I) as defined in claim 2, or a prodrug, tautomer or salt thereof, is administered in combination with at least one lipase inhibiting compound selected from the group of lipase inhibiting polymers, orlistat, panclicins, ATL-962 and lipstatin.

18. A method of treatment and/or prophylaxis according to any of the claims 12 to 17, characterized in that the CB₁ antagonistic compound, preferably the CB₁ antagonistic compound having formula (I) as defined in claim 2, or a prodrug, tautomer or salt thereof, is administered in a combination with the lipase inhibiting compound by simultaneous, separate or step-wise administration route.

19. Pharmaceutical product containing as a medicament a CB₁ antagonistic compound, preferably the CB₁ antagonistic compound having formula (I) as defined in claim 2, or a prodrug, tautomer or salt thereof, as a combination preparation with a lipase inhibiting compound for simultaneous, separate or step-wise administration in the treatment and/or prophylaxis of obesity.

20. Pharmaceutical product containing as a medicament a CB₁ antagonistic compound, preferably the CB₁ antagonistic compound having formula (I) as defined in claim 2, or a prodrug, tautomer or salt thereof, and a leaflet indicating that said CB₁ antagonistic compound may be administered in combination with a lipase inhibiting compound for simultaneous, separate or step-wise administration in the treatment and/or prophylaxis of obesity.

21. A CB₁ antagonistic compound, in particular a 4,5-dihydro-1H-pyrazole derivative which is a potent and selective antagonist of the cannabis CB₁-receptor, or a prodrug, tautomer or salt thereof, in combination with at least one lipase inhibiting compound.

22. A combination according to claim 21 of a CB₁ receptor antagonistic compound of formula (I), a prodrug thereof, a tautomer thereof or a salts thereof, in with at least one lipase inhibiting compound, 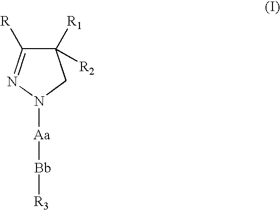
wherein
R and R₁ are the same or different and represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
Aa represents one of the groups (i), (ii), (iii), (iv) or (v) 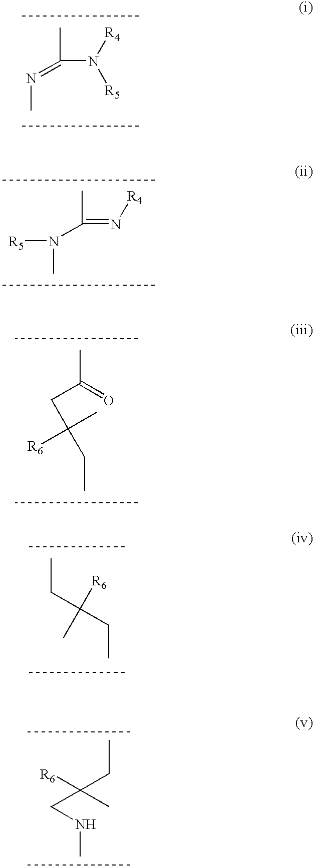
wherein
R₄ and R₅ independently of each other represent hydrogen or C₁₋₈ branched or unbranched alkyl or C₃₋₄ cycloalkyl or R₄ represents acetamido or dimethylamino or 2,2,2-trifluoroethyl or phenyl or pyridyl with the proviso that R₅ represents hydrogen
R₆ represents hydrogen or C₁₋₃ unbranched alkyl
Bb represents sulfonyl or carbonyl,
R₃ represents benzyl, phenyl, thienyl or pyridyl which may be substituted with 1, 2 or 3 substituents Y, which can be the same or different, or R₃ represents C₁₋₈ branched or unbranched alkyl or C₃₋₈ cycloalkyl, or R₃ represents naphtyl.

23. A combination according to claim 21, wherein in the compound having formula (I) R is the group 4-chlorophenyl, R₁ is phenyl, R₂ is hydrogen, Aa is the group (i) wherein R₄ is hydrogen and R₅ is methyl, Bb is sulfonyl, and R₃ represents 4-chlorophenyl, and salts thereof.

24. A combination according to claim 21, wherein the compound having formula (I) is a levorotatory enantiomer.

25. A combination according to anyone of the claims 21 to 24, wherein the CB₁ antagonistic compound is used in combination with at least one lipase inhibiting compound selected from the group of lipase inhibiting polymers, orlistat, panclicins, ATL-962 and lipstatin.
